(19) United States
(12) Patent Application Publication
(10) Pub. No.: US 2026/0054085 A1
(43) Pub. Date: Feb. 26, 2026

(54) PATCH NEGATIVE MAGNETIC DEVICE

(71) Applicant: Chih-Chiang HSU, New Taipei City (TW)

(72) Inventor: Chih-Chiang HSU, New Taipei City (TW)

(21) Appl. No.: 19/190,466

(22) Filed: Apr. 25, 2025

(30) Foreign Application Priority Data
Aug. 22, 2024 (TW) 113209062

(51) Int. Cl. A61N 2/12

(52) U.S. Cl. CPC A61N 2/12

(57) ABSTRACT

A patch negative magnetic device includes at least one supporting material and a motor piece. The supporting material has a plurality of array-arranged accommodating bags therein. At least one magnetic element is placed in the accommodating bag. The magnetic element has an S-pole surface and an N-pole surface. The supporting material is used to wrap around a body part of a human body. The S-pole surface of the magnetic element inside the supporting material faces the skin of the body part of the human body. The motor piece is combined with the surface of the supporting material to vibrate the magnetic element. Therefore, by vibrating and striking the supporting material, the negative magnetic field of the magnetic element can be strengthened to penetrate into the skin and muscles.

### BACKGROUND OF THE INVENTION

### 1. Field of the Invention

This invention relates to a patch negative magnetic device, in particular, to a patch negative magnetic patch device that surrounds a body part of a human body and can enhance the negative magnetic of the magnetic device through motor vibration for penetrating into the skin and muscles.

### 2. Description of the Prior Art

Dr. Albert Roy Davis, an American biophysicist, discovered that the cosmic magnetic poles have two completely different effects on living things. The positive magnetic field effect is positively charged, and has the effects such as oxidation, acidification, activation, excitement, and increased pressure. The negative magnetic field effect is negatively charged, and has the effects such as reduction, alkalinity, relaxation, calmness, and stress resistance.

The use of magnetic waves for health care was recorded as early as the Spring and Autumn Period. Bian Que once used magnets as pillows for Duke Mu of Qin and his concubines as the key to palace health care. Galen of ancient Greece in the second century AD and Avicenna, a famous Arab doctor in the eleventh century, both used magnets for their patients for health care. However, the practice of human magnetic medicine has always been based on the use of natural magnets. It was not until the end of the 19th century that artificial magnetic therapy equipment appeared in the West, and humans began to conduct in-depth and systematic research on magnetic medicine. In 1956, Japan commercialized magnets for use in conditioning, health care and other health-preserving purposes, with obvious results.

According to the Biot-Savart law, when the biomagnetic field of the human body adapts to the great magnetic field of the universe, it can maintain the normal physiology of the body's tissues and organs. Otherwise, abnormal reactions or illness will occur. Whenever a body part of the human body encounters different types of trauma, the electric and magnetic fields of that part are always positive. Through the mechanism of the meridians and nervous system, the body will concentrate the negative magnetic energy in the body to reach the affected part for the purpose of healing. Because there is no oxygen assistance in an acidic environment, the maintenance process must be in an alkaline environment in order to achieve good results.

When using magnets for health care, the area of the magnet must be larger than the area of the body part to be cared in order to have a better effect. In addition, there must be enough magnets and the intensity of the negative magnetic energy must be large enough to allow the user to be continuously exposed to the magnetic wave range. The intensity of the effect is sufficient to penetrate the skin and activate the body's own repair and maintenance mechanism to restore health and achieve a health-preserving effect.

However, if this type of magnet is to be used, the area is often too large or it is worn too much. Therefore, in the subject application, a motor piece is used in combination with a permanent magnet. The motor piece is combined with the surface of a supporting material with a permanent magnet inside to vibrate the magnetic element. By vibrating to strike the supporting material, the negative magnetic field of the magnetic element can be strengthened to penetrate the skin and muscles. Therefore, the subject application should be the best solution.

### SUMMARY OF THE INVENTION

The patch negative magnetic device of the present invention comprises at least one supporting material, which has a plurality of array-arranged accommodating bags inside, wherein at least one magnetic element is placed in the accommodating bag, and the magnetic element has an S-pole surface and an N-pole surface; and a motor piece, which is combined with the surface of the supporting material to vibrate the magnetic element.

More specifically, the supporting material is used to wrap around a body part of a human body, and the S-pole surface of the magnetic element inside the supporting material is facing a skin of the body part of the human body.

More specifically, the surface of the supporting material located on the skin of the body part of the human body is provided with at least one opening, and the opening is used to allow a local surface of the magnetic element placed inside the accommodating bag to be in direct contact with the skin of the body part of the human body.

More specifically, after the supporting material is wrapped around the body part of the human body, the S-pole surface or the N-pole surface of different magnetic elements can stick to and attract each other, so that the supporting material can be wrapped around and fixed on the body part of the human body.

More specifically, regarding the patch negative magnetic device, two or more supporting materials are able to attract each other so as to surround and fix on the body part of the human body.

More specifically, the supporting material is made of plastic, cloth or leather.

More specifically, the accommodating bag is of a sealed-type or an open-type.

More specifically, regarding the patch negative magnetic device, a rib is provided between every two accommodating bags, so as to separate magnetic elements at different positions.

More specifically, a plurality of accommodating bags are arranged in a grid pattern inside the supporting material.

More specifically, the magnetic element is a permanent magnet, which refers to a magnet that can maintain its magnetism for a long time.

More specifically, the magnetic element is circular or square.

More specifically, the motor piece is combined with the surface of the supporting material through a connecting member, and the connecting member is a magnetic element, a buckle element or a Velcro.

More specifically, the motor piece is an electric motor, a pneumatic motor or a hydraulic motor.

More specifically, the motor piece is further connected to a power piece.

### BRIEF DESCRIPTION OF THE DRAWINGS

FIG. 1A is a schematic diagram of the supporting material structure of the first embodiment of the patch negative magnetic device of the present invention.

FIG. 1B is a schematic diagram of the surface magnetism of the magnetic element of the patch negative magnetic device of the present invention.

FIG. 2A is a schematic diagram of the application of the first embodiment of the patch negative magnetic device of the present invention.

FIG. 2B is a schematic diagram of the application of the first embodiment of the patch negative magnetic device of the present invention.

FIG. 3 is a schematic diagram of the supporting material assembly structure of the second embodiment of the patch negative magnetic device of the present invention.

FIG. 4 is a schematic diagram of the application of the second embodiment of the patch negative magnetic device of the present invention.

FIG. 5 is a schematic diagram of the supporting material structure of the third embodiment of the patch negative magnetic device of the present invention.

### DETAILED DESCRIPTION OF THE INVENTION

Other technical contents, features and effects of the present invention will be clearly presented in the following detailed description of the preferred embodiments with reference to the drawings.

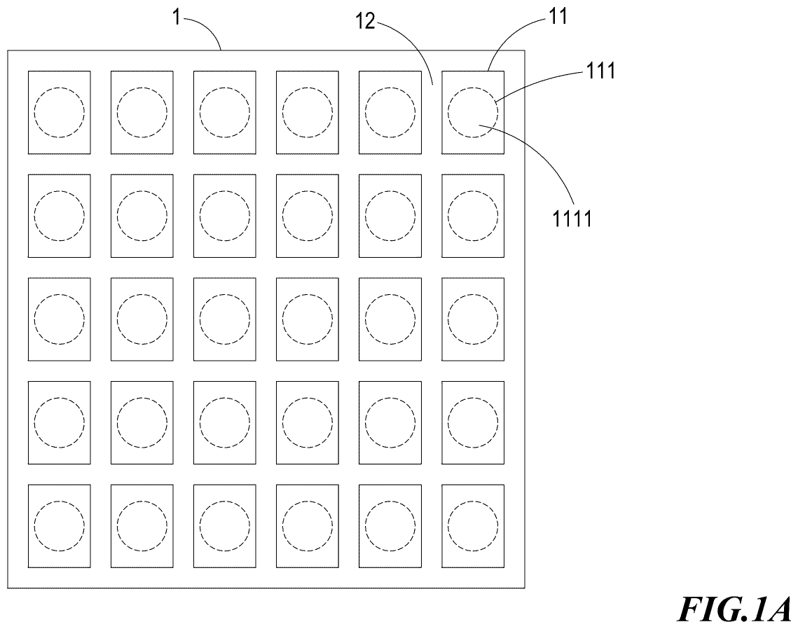

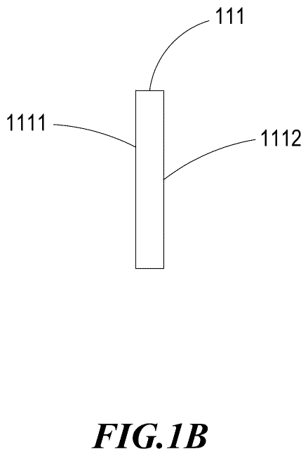

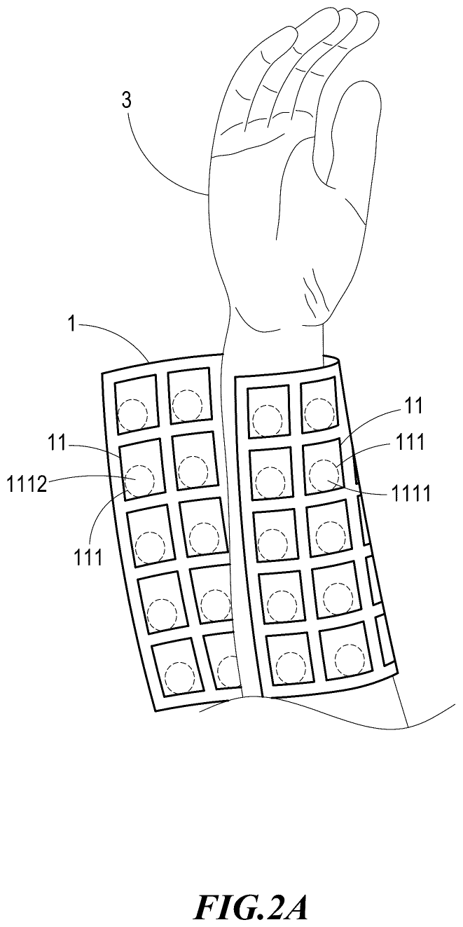

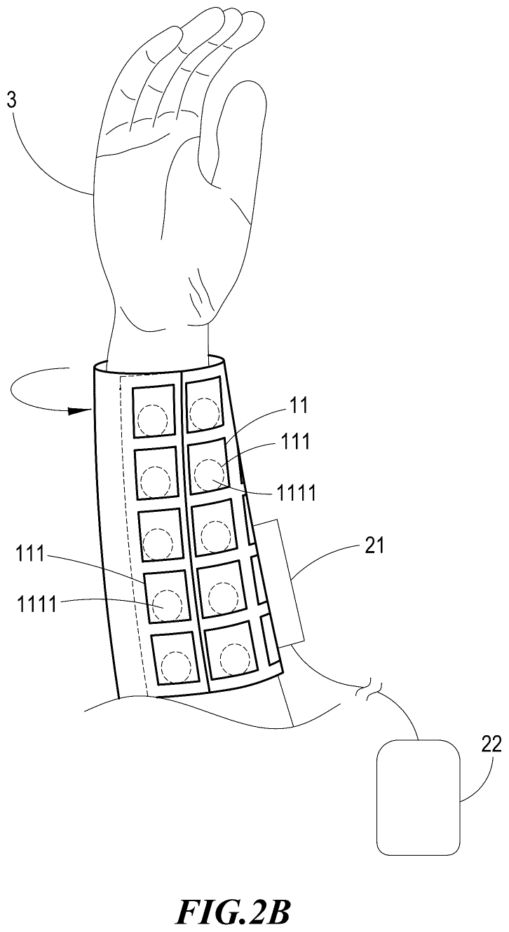

Please refer to FIGS. 1A, 1B, 2A and 2B, which are schematic diagrams of the supporting material structure of the first embodiment of the patch negative magnetic device of the present invention, a schematic diagram of the surface magnetism of the magnetic element, and an application schematic diagram of the first embodiment. As shown in the figures, the patch negative magnetic device includes at least one supporting material 1 and a motor piece 21.

As shown in FIG. 1A, the supporting material 1 has a plurality of array-arranged accommodating bags 11 inside. At least one magnetic element 111 is placed inside the accommodating bag 11. As shown in FIG. 1B, the magnetic element 111 has an S-pole surface 1112 and an N-pole surface 1111.

Among them, there is a rib 12 between every two accommodating bags 11. The rib 12 is used to separate the magnetic elements 111 at different positions to prevent the multiple magnetic elements 111 from being attracted to each other. The multiple ribs 12 can arrange the multiple accommodating bags 11 in a grid shape inside the supporting material 1.

Among them, the supporting material 1 is made of plastic, cloth or leather.

Among them, the magnetic element 111 is a permanent magnet, and the magnetic element 111 is round or square.

As shown in FIGS. 2A and 2B, the supporting material 1 can be used to wrap around a body part (arm 3) of a human body, and the S-pole surface 1112 of the magnetic element 111 inside the supporting material 1 is facing the skin of the body part (arm 3) of the human body. After the supporting material 1 wraps around the body part (arm 3) of the human body, the S-pole surfaces 1112 or N-pole surfaces 1111 of different magnetic elements 111 can stick to each other and be adsorbed. Accordingly, the supporting material 1 can be wrapped around and fixed on the body part (arm 3) of the human body.

Afterwards, as shown in FIG. 2B, the motor piece 21 is combined with the surface of the supporting material 1 to vibrate the magnetic element 111. In addition, the motor piece 21 is further connected to a power piece 22.

Among them, the power piece 22 is an external power supply, so that the motor piece 21 can be connected to a power supply cable, and the other end of the cable is connected to the external power supply.

Among them, the power piece 22 is a built-in power supply (any type of battery in the battery box, such as a dry cell, a lithium battery, a solar cell, or a lead-acid battery), which can be directly connected to the built-in power supply at the motor piece 21 or connected to the built-in power supply through a power supply wire. The built-in power supply can be further connected to a circuit board. The circuit board is used to control the power supply mechanism of the built-in power supply, so as to adjust the vibration time and/or vibration intensity of the motor piece 21 by adjusting the power supply time and/or power supply current.

The motor piece 21 is connected to the surface of the supporting material 1 through a connecting member. The connecting member is a magnetic element, a button element or a Velcro.

Among them, the motor piece 21 is an electric motor, a pneumatic motor or a hydraulic motor.

Among them, the motor piece 21 is used to expand the magnetic field function. By striking the magnetic element 111 inside the supporting material 1, the magnetic field is strengthened to penetrate into the skin and muscles.

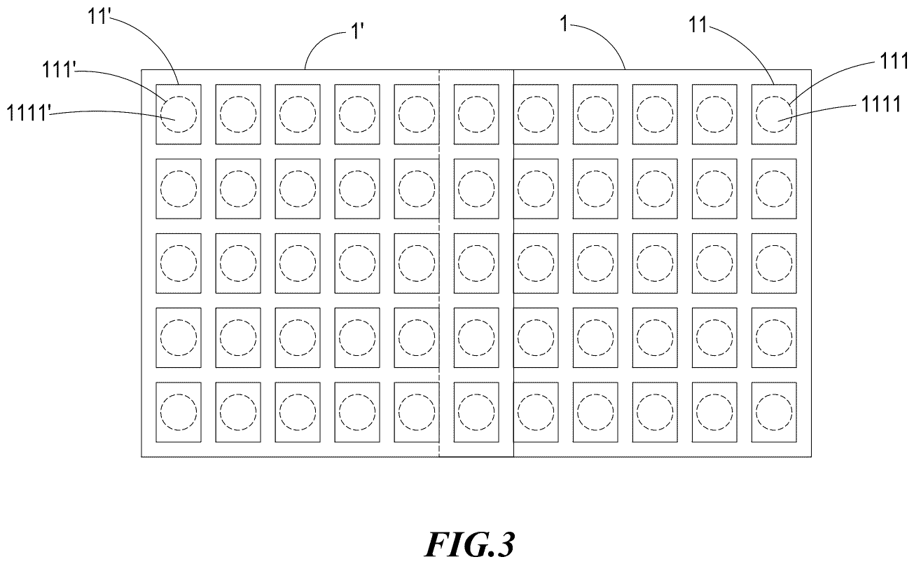

Since a single-piece supporting material 1 may not be sufficient to cover a body part of the human body, as shown in FIG. 3, at least two pieces of supporting materials 1,1′ can be stacked, wherein the supporting material 1′ also has a plurality of array-arranged accommodating bag 11′ inside. At least one magnetic element 111′ is placed inside the accommodating bag 11′, and the magnetic element 111′ has an S-pole surface and an N-pole surface 1111′.

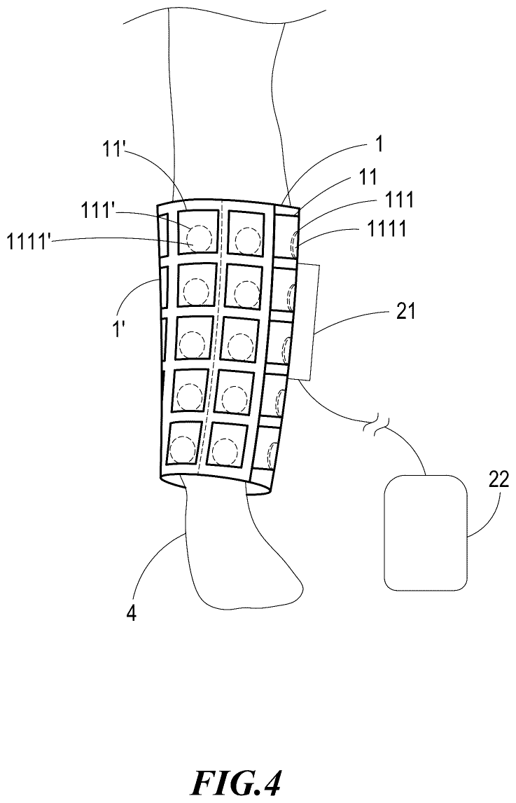

Therefore, the magnetic elements 111, 111′ of the supporting materials 1, 1′ can attract each other to increase the coverage range of the supporting materials. As shown in FIG. 4, after attaching the supporting materials 1, 1′ to each other, they are wrapped around and fixed on the body part (calf 4) of the human body. In addition to the calf mentioned in this embodiment, the supporting material can also wrap around other parts of the body, such as the head, neck, chest and back, abdomen and back, and buttocks.

Among them, the motor piece 21 is also combined on the surface of the supporting material 1, 1′. The motor piece 21 is used to vibrate the magnetic element 111. The vibration can not only increase the intensity of the negative magnetic field, but also enhance the circulation of the blood.

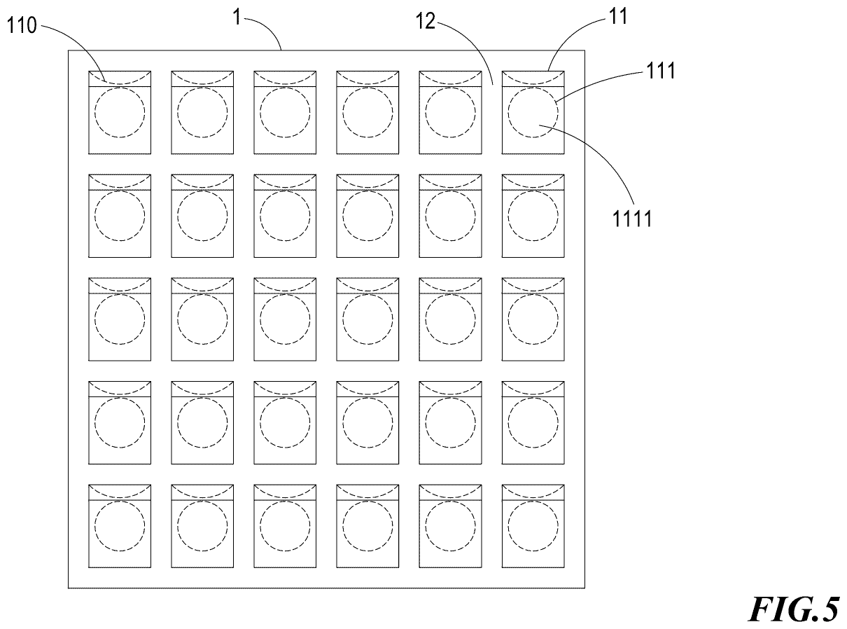

The supporting materials 1, 1′ described in the aforementioned FIG. 1A and FIG. 3 are both sealed-type, but the supporting material can also be designed to be open-type. As shown in FIG. 5, a bag opening 110 can be designed in the accommodating bag 11. By opening the bag opening 110, the magnetic element 111 inside the accommodating bag 11 can be replaced arbitrarily.

In addition, if it is an open-type, the magnetic element 111 inside the accommodating bag 11 can be arranged arbitrarily to enhance the negative magnetic strength of a specific part.

In addition, the surface of the supporting material is located on the skin of the body part of the human body and has at least one opening (bag opening 110). The opening is used to allow the local surface of the magnetic element placed inside the accommodating bag to be in direct contact with the skin of the body part of the human body. As shown in FIG. 5, the opening (bag opening 110) is located at the N pole. The N pole is not the surface facing the skin, and thus the opening (bag opening 110) can be opened at the N pole or at the S pole. That is, the opening (bag opening 110) can face towards or face behind the skin.

Compared with other conventional technologies, the advantages of the patch negative magnetic device provided by this invention are as follows:
(1) The motor of the subject application is used in association with a permanent magnet, and the motor piece component is combined with the surface of a negative magnetic device having a permanent magnet inside, so as to vibrate the magnetic element. Therefore, by vibrating and striking the negative magnetic device, the negative magnetic field of the magnetic element can be strengthened to penetrate into the skin and muscles.
(2) Because the subject application does not generate an electromagnetic field by driving magnets through power supply, it does not generate electromagnetic waves, and thus will not have any adverse effects on the human body.

Although the present invention has been disclosed in the form of embodiments, they are not intended to limit the present invention. Anyone with ordinary knowledge in the relevant technical field and understanding of the foregoing technical features and embodiments of the present invention may make slight changes and modifications without departing from the spirit and scope of the present invention. Therefore, the scope of protection of the present invention shall be subject to the scope of the claims attached to the specification herein.

What is claimed is:

1. A patch negative magnetic device, comprising:
at least one supporting material, having a plurality of array-arranged accommodating bags therein, wherein at least one magnetic element is placed in the accommodating bag, and the magnetic element has an S-pole surface and an N-pole surface; and
a motor piece, combined on a surface of the supporting material to vibrate the magnetic element.

2. The patch negative magnetic device according to claim 1, wherein the supporting material is used to wrap around a body part of a human body, and the S-pole surface of the magnetic element inside the supporting material is facing a skin of the body part of the human body.

3. The patch negative magnetic device according to claim 2, wherein the surface of the supporting material located on the skin of the body part of the human body is provided with at least one opening, and the opening is used to allow a local surface of the magnetic element placed inside the accommodating bag to be in direct contact with the skin of the body part of the human body.

4. The patch negative magnetic device according to claim 2, after the supporting material is wrapped around the body part of the human body, the S-pole surface or the N-pole surface of different magnetic elements can stick to and attract each other, so that the supporting material can be wrapped around and fixed on the body part of the human body.

5. The patch negative magnetic device according to claim 4, wherein two or more supporting materials are able to attract each other so as to surround and fix on the body part of the human body.

6. The patch negative magnetic device according to claim 1, wherein the supporting material is made of plastic, cloth or leather.

7. The patch negative magnetic device according to claim 1, wherein the accommodating bag is of a sealed-type or an open-type.

8. The patch negative magnetic device according to claim 1, wherein a rib is provided between each two accommodating bags to separate magnetic elements at different positions.

9. The patch negative magnetic device according to claim 1, wherein a plurality of accommodating bags are arranged in a grid pattern inside the supporting material.

10. The patch negative magnetic device according to claim 1, wherein the magnetic element is a permanent magnet.

11. The patch negative magnetic device according to claim 1, wherein the magnetic element is circular or square.

12. The patch negative magnetic device according to claim 1, wherein the motor piece is combined with the surface of the supporting material through a connecting member, and the connecting member is a magnetic element, a buckle element or a Velcro.

13. The patch negative magnetic device according to claim 1, wherein the motor piece is an electric motor, a pneumatic motor or a hydraulic motor.

14. The patch negative magnetic device according to claim 1, wherein the motor piece is further connected to a power piece.
